(19) United States
(12) Patent Application Publication
(10) Pub. No.: US 2012/0128838 A1
(43) Pub. Date: May 24, 2012

(54) SYSTEM AND METHOD FOR LEAN RECOVERY USING NON INVASIVE SENSORS

(75) Inventors: Manoj M. Virippil, Sioux City, IA (US); Daniel E. Tjaden, Dakota Dunes, SD (US)

(73) Assignee: TYSON FOODS, INC., Springdale, AR (US)

(21) Appl. No.: 13/325,567

(22) Filed: Dec. 14, 2011

Related U.S. Application Data
(63) Continuation-in-part of application No. 12/856,574, filed on Aug. 13, 2010.

(51) Int. Cl. G01N 33/12; G06K 9/00

(52) U.S. Cl. 426/231; 382/110

(57) ABSTRACT

An apparatus and method for non-invasive lean recovery from a sparse lean product, where the method can include conveying ground sparse lean product through a conveyance channel where the conveyance channel extends along a path that extends through a scanning position adjacent a scanner. The process includes scanning along a predetermined length with the scanner the ground sparse lean product traveling through the conveyance channel and further analyzing the scan and determining the percent fat content for each ground sparse lean product segment which is defined by the predetermined length of the ground sparse lean product within the volume of the conveyance channel and the cross section areas of the conveyance channel. The process can further include directing each ground sparse lean product segment down one of a plurality of processing paths corresponding to the one defined fat content range in which the corresponding percent fat content falls.

### CROSS REFERENCE TO RELATED APPLICATION

This application is a continuation-in-part of application Ser. No. 12/856,574 filed Aug. 13, 2010 entitled System and Method For Lean Recovery Using Non-Invasive Sensors, the entire disclosure of which is incorporated by reference herein.

### BACKGROUND OF INVENTION

1. Field of Invention

This invention relates generally to lean recovery and, more particularly, to lean recovery using sensors.

2. Background Art

Attention within the meat industry has been drawn to the dangers of high-fat diets, including correlations made to an increased incidence of cardiovascular diseases, such as coronary heart disease and arteriosclerosis. As a consequence, the medical profession has suggested that the consumption of fat should be reduced. One way to accomplish this is to eat meats that have been processed so that they contain a reduced fat content.

One method to reduce the percent fat content in meat is simply to manually cut fat from the meat, which is commonly referred to as trimming the meat. Portions of meat having higher amounts of fat is cut or trimmed away from the attached portions of meat having a lower amount of fat (meat that is more lean). The trimmings are separated by operators with sharp cutting utensils. However, manually cutting away the more fatty portion from the more lean portions, is not effective in reducing the fat content of the remaining more lean portions to a percent fat content lower than about five percent. In addition, this process does not assist in recovering any further lower fat lean portions from the trimmings. Further, skilled workers and time are required to cut the meat, thus making the process expensive and inefficient, further necessitating the need to recover usable lean from the trimmings.

In an attempt to reduce the fat content of meat and meat trimmings other processes have been proposed and utilized. These processes typically employ one or more of the following approaches. First, the fat can be extracted from meat by mechanical techniques, such as by the use of a grinder, a crusher, a press, a comminutor, or a micro-comminutor. These procedures have been employed with or without accompanying high temperatures. Physical extraction techniques have also been utilized, such as the use of heat, and reaction of gases with meats, including fluid extraction. Fat has also been removed employing chemical extraction techniques, such as the use of chemical reagents, including acids.

Unfortunately, these techniques generally have a detrimental impact on the meat or alter the meat's protein profile, vitamin profile, color, texture and/or water content. For example, high temperatures denature meat. The use of diluents, such as water, can leach water-soluble proteins and vitamins from the meat and can increase the moisture content of the defatted product. Additionally, when diluents are used with micro-comminution of meat, the functional properties of the resulting product can be adversely affected. The use of chemical reagents, acid or alkaline treatment of meat facilitates the binding of anions or cations, respectively, to the protein, thereby adversely affecting the meat's properties.

Moreover, it is often the subsequent separation step that is critical to the success or failure of a defatting process. Even if a substantial amount of fat is initially liberated from the meat, unless the fat is effectively separated from the meat, the process will not be a success. For example, even if the proper choice of conditions for grinding or comminuting meat produces a substantial fat-containing fraction, conventional devices, such as conventional decanter centrifuges, are not completely effective in separating the resulting fractions.

Decanter centrifuge methods have also been utilized for producing lower fat lean meat having substantially the same functionality, protein profile, vitamin profile, color, texture and water content as the raw meat starting material. The reduced fat meat, however, can often contain from about 0% to 10% fat and can have a substantially reduced level of cholesterol. The decanter centrifuge can have a hollow, centrifugal rotor with a longitudinal axis of rotation a. The centrifugal rotor defines a generally cylindrical bowl tapered at one end to form a beach. The centrifuge also can have a feed tube for introducing starting material into a delivery zone in the interior of the cylindrical bowl and a fluid inlet tube for proportionately metering a fluid into the feed tube. A screw conveyor, can be disposed in the cylindrical bowl to cause a substantially solid portions to be discharged out of at least one solid discharge port located at the tapered end of the rotor and a substantially liquid fraction to be discharged out of at least one liquid discharge port located at the opposing end of the rotor.

Further, Low temperature rendering processes have been used to separate protein from fatty tissue in animal trimmings. The processes generally involve comminuting fatty tissue from animals, such as hogs or cattle, to form a semi-solid slurry or meat emulsion, heating the slurry or emulsion to melt the fat, and then separating the fat and protein by centrifugation. The protein can then be used as an ingredient in processed meat products such as sausage and other cured and processed meats. It has been found that the protein or meat provided by prior art low temperature rendering processes suffer from undesirable flavor changes shortly after production. In order to reduce the flavor changes after low temperature rendering processes, some processes use conditioning agents which reacts or combines with the pigments of the meat to reduce the activity of the pigments which catalyzes the development of off-flavor.

The government provides that a certain quality of meat product obtained from animal trimmings can be used undeclared in meat products of the same species. For example, “finely textured beef” and “lean finely textured beef” can be used in ground beef without being declared on the label, however there may be a percentage limitation for the amount added. “Finely textured meat” is required to have a fat content of less than a defined percent; a protein content of greater than a defined percent. “Lean finely textured meat” is required to have a fat content of less than a defined percent, by weight, and complies with the other requirements of “finely textured meat.” A low temperature rendering process can include the process steps of: heating desinewed animal trimmings in a heat exchanger having a first-in and first-out arrangement to provide heating of the desinewed animal trimmings to a temperature in the range of about 90.degree. F. to about 120.degree. F. to form a heated slurry; separating a solids stream and a liquids stream from the heated slurry, the solids stream containing an increased weight percent of protein and moisture compared with the weight percent of protein and moisture in the heated slurry, and the liquids stream containing an increased weight percent of tallow compared with the weight percent of tallow in the heated slurry; separating a heavy phase and a light phase from the liquids stream.

The step of separating a solids stream and a liquids stream from the heated slurry can occur in a decanter, and the step of separating a heavy phase and a light phase from the liquids stream can occur in a centrifuge, and the meat product can be frozen within about 30 minutes of heating the desinewed animal trimmings in a heat exchanger.

In contrast, testing may be performed in a noninvasive manner through the use of sensors, such as microwave sensors. These provide a valuable improvement in monitoring meat flows. However, heretofore microwave sensors have not been required to monitor very low-fat raw lean meat supplies. It has been discovered that such microwave sensor equipment typically is not adequate to consistently monitor these very low-fat meat supplies. More particularly, it has been discovered that the sensitivity of this equipment to temperature variations renders it unreliable for a very low fat application. However, methods have been used for calibrating microwave sensors for measurement of meat fat, protein, and moisture content and further separating portions of the meat that exceed the standard fat, protein, and moisture content.

Temperature calibrating alleviates a persistent erroneous measurement problem which developed in attempting to use available equipment for measuring very low levels of meat parameters. The sensing method can be utilized in a method of separating meat products into multiple flows, at least one flow having a meat parameter in excess of a predetermined amount. Such methods can include the steps of providing a microwave sensor unit having a location at which microwave power is applied; flowing a supply of meat through the microwave sensor unit; applying microwave power of the microwave sensor unit to the flowing supply of meat to generate microwave signal readings of the meat products; sensing the temperature of the flowing supply of meat to generate a temperature signal reading; transmitting the microwave signal readings and the temperature signal reading to a processor of the microwave sensor unit; processing the microwave signal readings and the temperature signal reading together with a preloaded set of temperature calibration coefficients in order to generate temperature corrected meat parameter value outputs for the microwave sensor unit for variations in temperature of the flowing supply of meat; comparing the meat parameter derived during the processing step with a predetermined meat parameter value; and diverting from the flowing supply of meat a portion thereof which had been determined during the processing step to have a meat parameter in excess of said predetermined amount thereby separating out product having lower fat content. However, this process is not useful for lean recovery from meat having higher fat content.

Amore effective method for lean recovery is needed to resolve the short comings of previous methods.

### BRIEF SUMMARY OF INVENTION

The invention is method and system for segregating sparse lean product based on percent fat content. One embodiment of the invention is a method including grinding a sparse lean product into a ground sparse lean product and outputting the ground sparse lean product through a conveyance channel from and entry end to an exit end. The process includes extending the conveyance channel having the ground sparse lean traveling there through by pushing the product through the conveyance channel along a path that extends through a scanning position adjacent a scanner. The process includes scanning along a predetermined length with the scanner the ground sparse lean product traveling through the conveyance channel as the ground sparse lean product passes through the scanning position and further analyzing the scan and determining the percent fat content for each ground sparse lean product segment which is defined by the predetermined length of the ground sparse lean product within the volume of the conveyance channel and the cross section areas of the conveyance channel occupied by the ground sparse lean product at the scanning position.

The process can further include cutting away each ground sparse lean product segment and sorting based on which of a plurality of defined fat content ranges the corresponding percent fat content is within and directing each ground sparse lean product segment down one of a plurality of processing paths corresponding to the one defined fat content range in which the corresponding percent fat content falls. The scanner can be one of many types of comparable scanners including an X-Ray scanner, a near infra-Red scanner, an ultra violet scanner, a guided microwave spectroscopy system and other appropriate scanning tools. The scanner can capture scan data in incremental segments, by scanning incrementally, segments of the sparse lean product flowing or traveling through the conveyance channel, where the segments are defined by an optimal predetermined length. The scan data for each scan segment can be captured and analyzed for percent fat content. The predetermined length scanned can be from about approximately 4 mm to about approximately 10 mm. The plurality of processing paths can be a plurality of conveyor lanes.

The conveyance channels can be tubes or other method of conveyance of the ground sparse lean product, and the cross section areas of the conveyance channel occupied by the ground sparse lean product can be the cross section areas of the tube occupied by the ground sparse lean product and the exit end can be an exit end of the tube, which is communicably linked to a plurality of exit tubes. Each of the exit tubes can have a knife gate adapted to selectively open and close as can be activated by a solenoid valve push mechanism or other comparable mechanism. The knife gate can be before or after the analytical tool.

A controller having a processor function can receive flow data representative of the product flow through the tubing. The flow data can be received from flow sensors place along the length of the tubing. The flow data transmitted to the controller can include the rate of flow of the product through the tubing. This data can be utilized by the controller to control the actuation of the cutter at the appropriate time. An encoder in electronic signal communication with the scanner and the solenoid valve push mechanism can be utilized such that the scanner is adapted to provide an electronic control signal that can be utilized to control the solenoid valve push mechanism to selectively open and close the knife gate based on the rate of flow of the product, the predetermined scan length, which is correlated to a percent fat content percent fat content.

The sparse lean product segments can undergo further scanning of each sparse lean product segment for segment fat content and further segregation of each sparse lean product segment further based on the segment's fat content. The sparse lean product segments can also be further processed by recombining a combination of sparse lean product segments to achieve a desired recombined percent fat content.

Another embodiment of the invention is a system for segregating sparse lean product based on percent fact content. The system can include a grinder having a pre-sized reduction end plate adapted to mince or dice a sparse lean product into a ground sparse lean product. An output port can be communicably attached to an output of the pre-sized end plate and said output port can be mounted as a conduit to receive the minced sparse lean product from the output of the end plate and channel the minced or ground sparse lean product into a conveyor channel where said conveyor channel can extend from an entry end to an exit end along a path that extends through a scanning position.

A scanner can be utilized that is operable to scan along a predetermined length the ground sparse lean product traveling through the conveyance channel as the ground sparse lean product passes through the scanning position and to generate scan data representative of the fat content. A processor can be electronically integrated with said scanner and operable to analyze the scan data and determine the percent fat content for each ground sparse lean product segment which is defined by the predetermined length of the ground sparse lean product within the volume of the conveyance channel and the cross section areas of the conveyance channel occupied by the ground sparse lean product at the scanning position. A cutter attached proximate the exit end of the conveyance channel can be utilized to cut away each incremental ground sparse lean product segment.

An encoder can be electronically integrated with the processor and the cutter and operable to control the cutter to open and cut based on which of a plurality of defined fat content ranges the corresponding percent fat content is within. There can be processing paths each corresponding to the one defined fat content range in which the corresponding percent fat content falls.

The system can utilize a scanner that is an X-Ray scanner, a near infra-Red scanner, ultra-violet scanner, guided microwave spectroscopy system or other appropriate scanning tools. Although other types of comparable scanners can be utilized that are operable to capture scan data representative of the fat content and can be analyzed and interpreted. The predetermined length can be from about 4 mm to about 10 mm, which is an achievable resolution and sufficient for determining fat content.

The system can utilize conveyance channels that are tubes, and therefore, the cross section areas of the conveyance channel occupied by the ground sparse lean product is the cross section areas of the tube occupied by the ground sparse lean product and the exit end is an exit end of the tube, which is communicably linked to a plurality of exit tubes. The system can be designed where each of the exit tubes have a knife gate adapted to selectively open and close as activated by a solenoid valve push mechanism. The encoder in electronic signal communication with the processor of the scanner and the solenoid valve push mechanism can be adapted such that the scanner is adapted to provide an electronic control signal to control the solenoid valve push mechanism to selectively open and close the knife gate based on the product flow, predetermined segment length, which corresponds to a percent fat content.

The system can further comprising a second scanner operable to scan each sparse lean product segment for the segment's fat content; and a segregator operable for segregating each sparse lean product segment further based on the segment fat content. The system can also include a combiner operable for recombining a combination of sparse lean product segments to achieve a recombined product to achieve a desired recombined fat content. The invention provides a non-invasive method to accurately recover lean from sparse lean products.

It should be noted that the embodiments of the present invention described and claimed herein primarily show the cutters positioned after the scanner in order to cut the previously scanned product into segments that correspond to previously scanned product segments have a percentage fat content. However, another embodiment that could be utilized without departing from the scope of the invention described herein, is to position the cutter prior the scanner such that the cutters cut the product into predetermined sized segments, which are subsequently scanned for percent fat content and sorted accordingly.

An alternative to the grinder/reducer and conveyance channel tube combination could be a pre-sizer/reducer, which can reduce the sparse lean to presized chunks, which can be output to a flighted conveyor having flights timed to the output of the pre-sizer such that the presized chunks will fall between the flights. The scanner can be positioned to scan the fat content of the chunks between the flights. The scan data can be captured by a controller and utilized to control a reject or sorting device.

The invention as described herein provides a system and method to accurately and consistently control the percent fat content in a meat product, which is accomplished by accurately measuring the percent fat content of a optimally sized segment of meat whereby the percent content of the meat segment can be determined accurately and where the meat segments can be sorted based on the determined percent fat content and the meat segments can be further scanned and sorted to refine the accuracy. This provides a system and method for accurately controlling fat content without changing the characteristics or appearance of the meat. Although, the term “sparse lean” is used throughout the specification when describing the product that is being operated on by the invention described and claimed herein, the invention can be utilized for lean recovery irrespective of the percentage fat content of the product, thus the term “sparse lean” does not limit the scope of the invention or its utility in any way and is used in a very broad sense. Further, the invention can be utilized for any species of lean product, including but not limited to beef, pork, lamb, and venison. The terms “conveyance” or “conveyance channel” are used throughout the specification and is utilized to mean an act of conveying, or means of conveying, means of carrying, transporting or transferring from one position to another.

These and other advantageous features of the present invention will be in part apparent and in part pointed out herein below.

### BRIEF DESCRIPTION OF THE DRAWINGS

For a better understanding of the present invention, reference may be made to the accompanying drawings in which:

FIG. 1 is a top view of the lean recovery system;

FIG. 2 is a side view of the lean recovery system;

FIG. 3 is a front view of the lean recovery system is shown;

FIG. 4 is a perspective view of the lean recovery system;

FIG. 5 is a perspective view of the grinder, conveyor and cutter interface.

FIG. 6, is a perspective view of the cutter and exit tube interfaces.

FIG. 7, is an illustration of the controller station;

FIG. 8 is an illustration of the system and method;

FIG. 9 is an illustration of the system having a flighted conveyor;

FIG. 10 is an illustration of the system flighted conveyor and reject mechanism;

FIG. 11 is an illustration of the system cutter, and flighted conveyor;

FIG. 12 is an illustration of a system with rotary barrel; and

FIG. 13 is a side view illustration of as system with a rotary barrel.

While the invention is susceptible to various modifications and alternative forms, specific embodiments thereof are shown by way of example in the drawings and will herein be described in detail. It should be understood, however, that the drawings and detailed description presented herein are not intended to limit the invention to the particular embodiment disclosed, but on the contrary, the intention is to cover all modifications, equivalents, and alternatives falling within the spirit and scope of the present invention as defined by the appended claims.

### DETAILED DESCRIPTION OF INVENTION

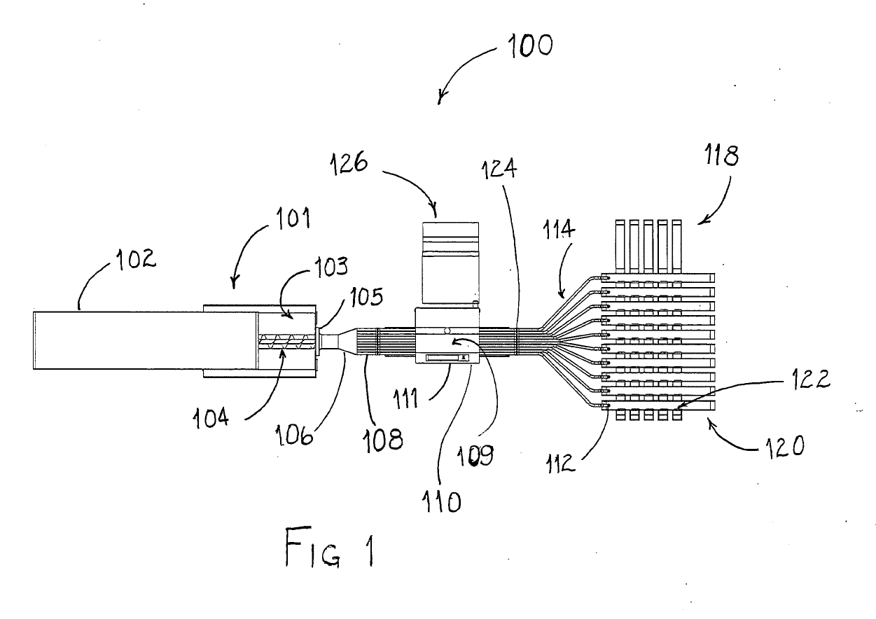

According to the embodiment(s) of the present invention, various views are illustrated in FIG. 1-13 and like reference numerals are being used consistently throughout to refer to like and corresponding parts of the invention for all of the various views and figures of the drawing. Also, please note that the first digit(s) of the reference number for a given item or part of the invention should correspond to the Fig. number in which the item or part is first identified. Further, this application incorporates by reference in its entirety, application Ser. No. 12/856,574 filed Aug. 13, 2010 entitled System and Method For Lean Recovery Using Non-Invasive Sensors.

One embodiment of the present invention comprising a grinder, conveyor, and cutter teaches a novel apparatus and method for recovering and segregating lean from sparse lean product based on percent fat content. Sparse lean product or trim can be accumulated trim in an auger/grinder. The grinder can be equipped with rotating knife or cutting plate to pre-size the product. The auger/grinder's end plate can be adapted with an adapter to output the ground product horizontally. A conveyance system can be communicably connected to the output of the grinder (an example of a conveyance system can be a plurality of tubes) for conveying the ground product to an X-ray station disposed after the output of the auger/grinder. An X-ray, Near Infra-Red (NIR), Ultra-Violet, guided microwave spectroscopy system or other appropriate scanning system can be used to scan and detect fat content scan data in order to determine the combined fat analysis (FA) by every predefined scan length of the ground sparse lean product within the tube cross section. The X-ray or NIR can provide an input to a controller system that can at the appropriate time activate the solenoid valve push mechanism to open/close a knife gate. The cut product will be dropped onto a conveyor that transports the product to a combining process or other process. The X-ray or NIR or other appropriate scanning tool software along with the controller can keep the aggregate FA of each of the products/conveyors (output streams). Each of the output streams can further be combined to get a desired out fat % combination.

The details of the invention and various embodiments can be better understood by referring to the figures of the drawing. Referring to FIG. 1, a top view of the lean recovery system is shown. The top view shows the end to end process from the input of the sparse lean product to the recovered lean product segments that have been sorted for further processing. The sparse lean product or meat trimmings can be input into the system by the sparse lean feed conveyor 102. The sparse lean product or trimmings can be conveyed from the input end of the conveyor to the output end of the conveyor where it can be dropped into a reducer 101 whereby the sparse lean product can be diced or reduced into smaller pieces. The reducer 101 is illustrated in this figure as a grinder system including a grinder hopper 103 which channels the sparse lean product into the grinder 104. The grinder can be equipped with a rotating knife or auger type mechanism to pre-size the product and the grinder can have a pre-sizer end plate that is modified to output the minced or diced product horizontally. Other types of pre-size reducers can be utilized that are well known in the art without departing from the scope of the invention.

The grinder 104 can have a horizontal adaptor output 105 which can channel the minced product into a conduit 106 which in turn channels the product through tubing 108. The tubing 108 can extend through a scanner 110 having an internal scanner position 109 whereby the scanner can scan incremental lengths of the product traveling through the tubing passing through the scanner position. The scanner 110 can have a scanner control interface 111 whereby a user can provide inputs into the scanner as well as monitor various operational parameters. The scanner can be adapted to scan the product along incremental predetermined lengths to thereby scan incremental segments of the ground sparse lean product traveling through the conveyance channel illustrated in this figure as tubing.

The incremental predetermined lengths of ground sparse lean product can be scanned as the product passes through the scan position. The scanner can be an x-ray type scanner, a near infrared type (NIR) scanner, an Ultra-Violet scanner, guided microwave spectroscopy system or other appropriate scanning tool, which can be adapted to scan the product traveling through the conveyance channel and take the scanned data and analyze it to determine the fat content. The scanner can be adapted to scan incremental lengths and determine the fat content contained within those incremental lengths in order to more accurately account for changes in fat content of the ground sparse lean product traveling through the conveyance channel. The scanning function can be implemented in various different ways without departing from the scope of the invention, for example the scanner could be position after the knife gate for sorting after a cut is made.

One embodiment of the present invention can include a scanner that scans over incremental predetermined lengths where the lengths are from about approximately 4 millimeters to about approximately 10 millimeters in length. The non-invasive lean recovery system 100 can also include a controller 126 that communicates with the scanner 110 for exchange of control parameters, such as percent fat content for a given segment. The controller 126 can also communicate with and control the conveyor 102, the reducer 101, the scanner 110, the various product flow sensors 124, the cutters 112, the product conveyors 118, the reject conveyors 120, and the various rejection mechanisms 122. The controller could be at a remote location and communicably linked to the scanner, knife gates and other devices.

Once the ground sparse lean product has traveled through the portion of a conveyance channel or tubing that extends through the scanner, the ground sparse lean product can continue to travel through the conveyance channel through the reject portion of the tubing or conveyance channel 114. The reject portion of the conveyance channel 114 will extend to cutters 112 which will incrementally cut away segments of the ground sparse lean product where the incremental segments are cut at lengths consistent with the predetermined length of the scan. Therefore each ground sparse lean product segment that is cut away will have a corresponding percent fat content that has been calculated by the scanner.

Each ground sparse lean product segment that has been cut away will be transferred to a reject conveyor 120 and as the ground sparse lean product segment travels along the reject conveyor the product segment can be selectively sorted based on its percent fat content. This can be accomplished by utilizing ejection mechanisms 122 spaced incrementally along the length of the reject conveyor 120. The ejection mechanism can laterally push the product segment off of the reject conveyor 120 to fall down a chute that channels the segment to an appropriate product conveyor that corresponds to a predefined percent of fat content percentage range. Therefore, for each product segment that is cut away a fat content for that segment has been determined by the scanner. The fat content determined can fall within various predetermined percent fat content percentage ranges and the product segments can be sorted in accordance to those predetermined percentage ranges.

A plurality of product conveyors can be utilized whereby each of the plurality of conveyors can correspond to a given percentage range. The product segment can be in turn ejected at the appropriate time to fall on the appropriate corresponding product conveyor having a predefined percentage range for which the fat content of the product segment falls. In FIG. 1 the reject conveyor 120 and the product conveyor 118 are illustrated as a plurality of endless belt conveyors that travel in parallel. However, other conveyance means can be utilized. The ejection mechanism is shown in this figure as a plurality of air nozzles that are incrementally spaced along a length of a reject conveyor whereby the air nozzles are designed and controlled to emit an air jet pulse to laterally push a product segment off the reject conveyor into the appropriate chute channeling the product segment to the appropriate product conveyor.

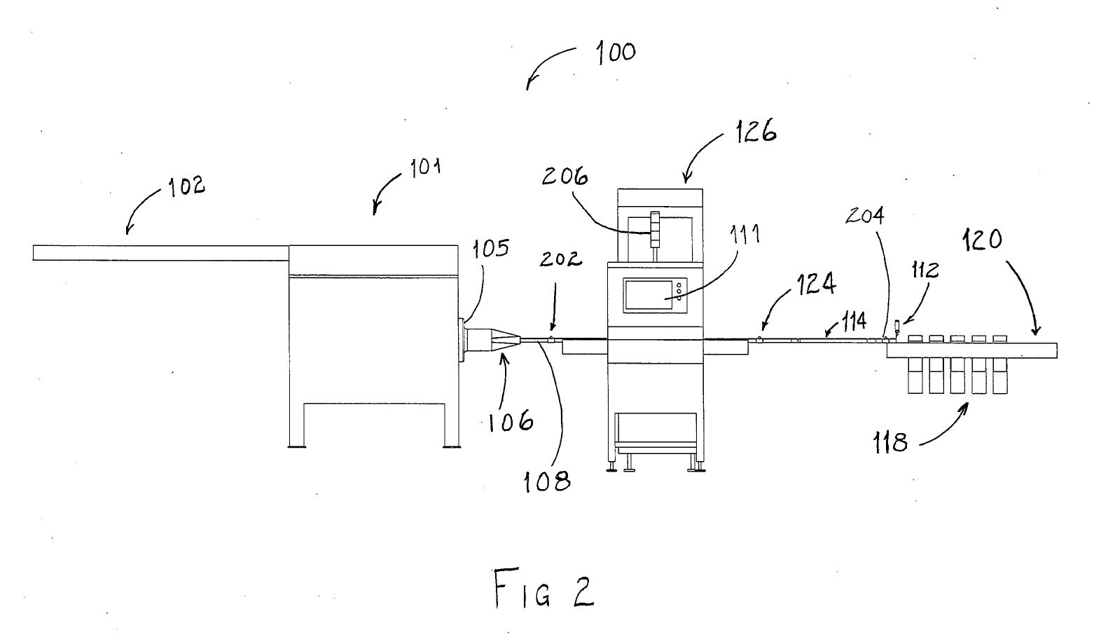

Referring to FIG. 2, a side view of the lean recovery system 100 is shown. Again an end to end view of the lean recovery system is shown. As illustrated previously the sparse lean feed conveyor 102 is positioned to convey the sparse lean product or trimmings to the reducer 101. The reducer 101 is designed to flow the product through the horizontal adaptor output plate 105 and into the conduit 106 and further into a plurality of conveyance channels 108. Product flow sensors can be positioned along the conveyance channel in order to determine the product flow at various positions along the channel. The product flow sensors are shown in FIG. 2 as items 202, 124, and 204. This side view reveals the scanner control interface 111 whereby a user can provide certain inputs and view certain operational parameters of the scanner. The scanner can have a stack light 206 than can provide an indication of the operation mode of the scanner. The controller 126 is again shown adjacent to the scanner. The reject tubing 114 is shown extending into a cutter 112 which is positioned above the product end reject conveyors.

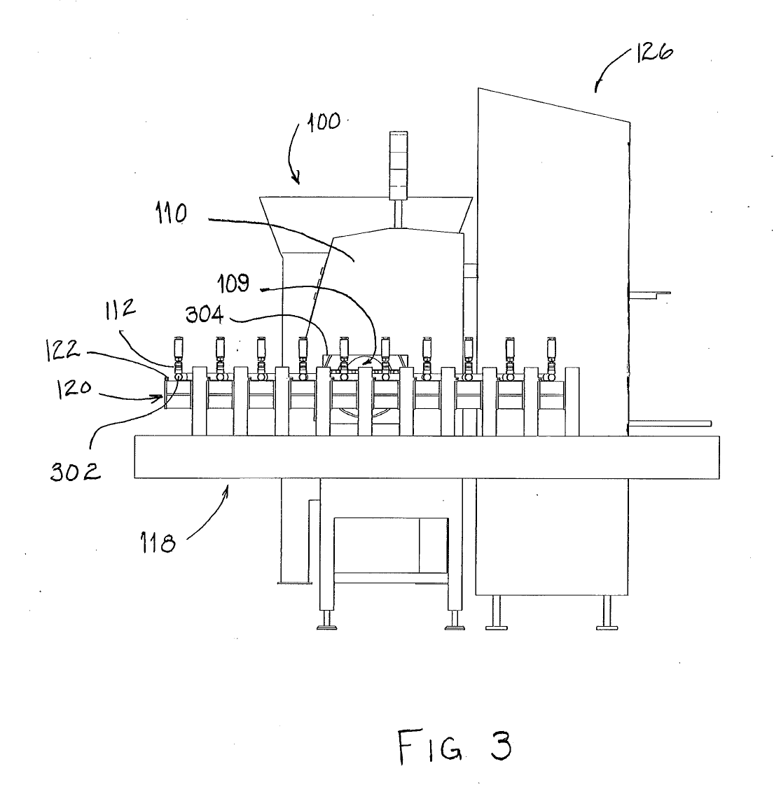

Referring to FIG. 3, a front view of the lean recovery system 100 is shown. Again the scanner 110 and the controller 126 are shown adjacent with a communication conduit connecting the two panels. This front view reveals the scanner chamber 304 in which there is a scan position 109 and it is at this position where the scanner scans the product over a predetermined length. This view also shows the view of the cutters 112 having a conduit 302 out of which the product segments exit to thereby fall onto the reject conveyor 120. The reject conveyor can be designed to transfer any rejected product not meeting one of the predetermined percentage fat content ranges onto a take away conveyor (not shown) for further processing. Any product selectively ejected based on falling within one of the predetermined ranges for percent fat content will be ejected to one of the plurality of product conveyors 118.

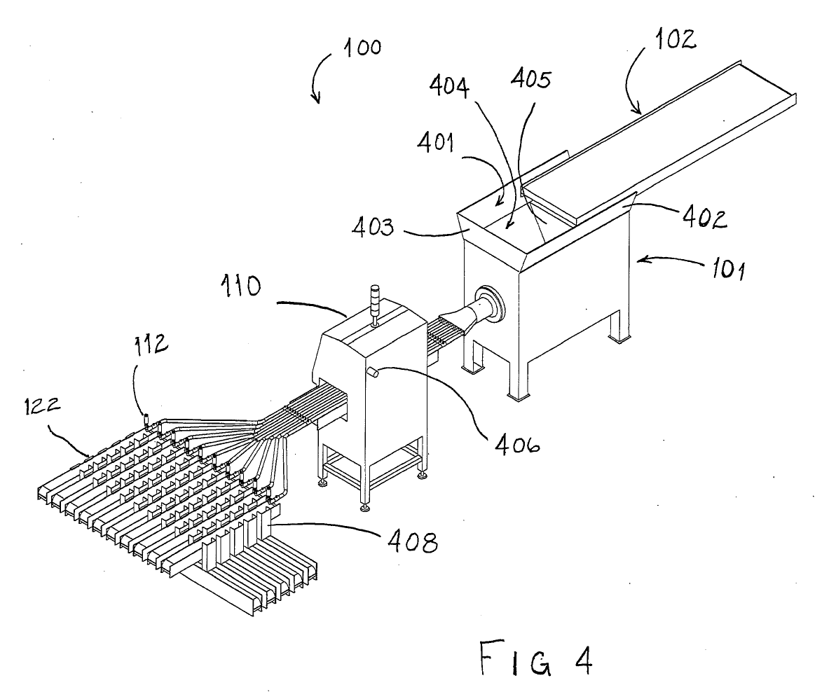

Referring to FIG. 4, a perspective view of the lean recovery system is shown. The sparse lean feed conveyor 102 is shown as an endless belt conveyor that drops the sparse lean product onto hopper deflector plates 401, 402, 403, 404, and 405. The deflector plates channel the product into the reducer. A communication conduit 406 is shown in this view that can be connected to the controller 126 (not shown in this view). As previously indicated the cutter 112 can cut the ground sparse lean product into product segments having a length that corresponds to the predetermined scan length and the product segments can fall onto the reject conveyor where the product segments can be selectively ejected down a chute 408 which will direct the product segment to fall on the selected product conveyor having a percentage fat content range that corresponds to the fat content of the product segment determined by the scanner.

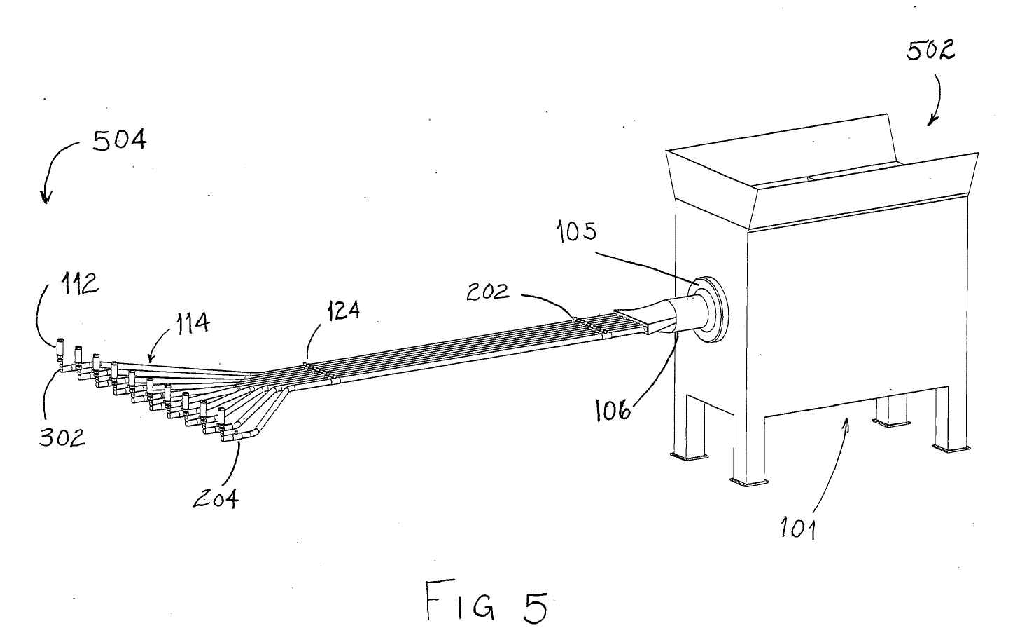

Referring to FIG. 5, a perspective view of the cutter and reject tubing and product conveyor channels are shown interfacing with the grinder. In this view the grinder is shown having a grinder input end 502 through which the sparse lean product can be inserted and subsequently dropped and directed by the hopper deflector plates. As previously indicated products flow sensors 124, 202, and 204 can be utilized to determine the product flow along various positions of the product conveyor channels. Therefore, the sparse lean product is input through the grinder input end 502 and is minced or diced into a ground sparse lean product that travels through the conveyance channels and is ultimately cut by the cutters 112 and the product segments are output through the cutter conduits 302.

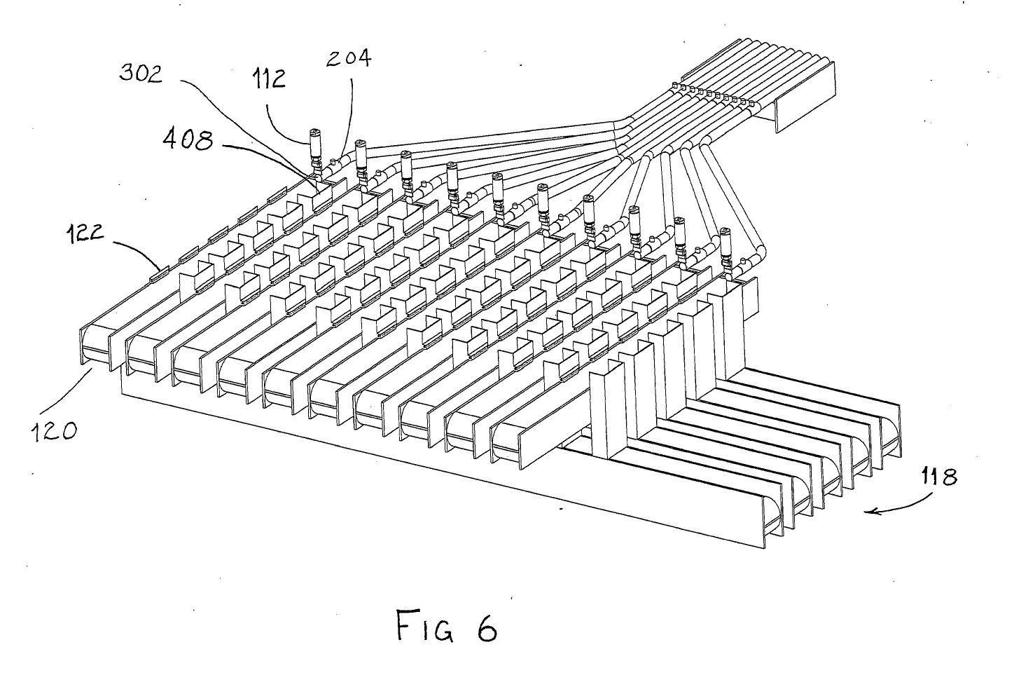

Referring to FIG. 6, a perspective view of the cutter and reject tubing 114 is shown. Again the cutters 112 will cut away product segments which will in turn fall onto the reject conveyors 120. As the product segments travel along the reject conveyors 120 the product segments can be ejected laterally off of the reject conveyor by an ejection mechanism 122 which in this case is shown as a air nozzle that is controlled by the controller to selectively emit an air jet puff to laterally push the product segment laterally off of the reject conveyor down a chute 408 which will channel it to the appropriate product conveyor 118. Each of the cutters and the conveyors can be communicably linked to the controller 126 such that the controller can provide control parameters or inputs to thereby control the operation of the various components. Further the product flow sensors 204 and the other product flow sensors as mentioned previously, can be communicably linked with the controller to provide product flow data which cab be utilized to control the various components of the system. The communication links between the controller and the various other components of the system can be wireless or hardwire. Either means of communication is well known and can be readily implemented by one skilled in the art.

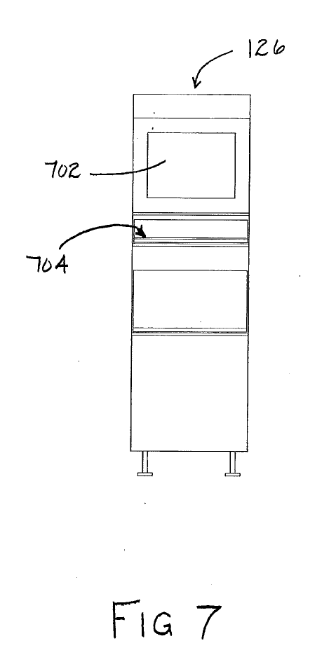

Referring to FIG. 7, an illustration of the controller station as it is shown. The controller station 126 is shown having a display 702 whereby a user can view various inputs and control parameters and the various subcomponent system operations such as the operation of the grinder as well as viewing product flow data provided by the product flow sensors. The controller 126 can also have an input device 704 that allows the user to input certain control parameters or control functions for controlling the operation of the overall system.

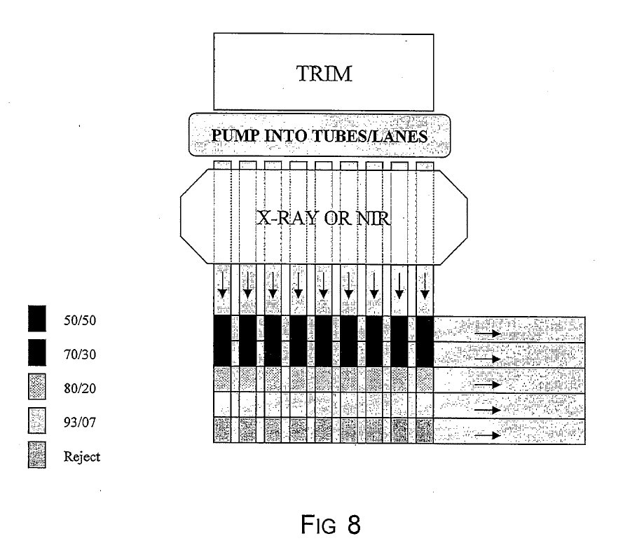

Referring to FIG. 8, an illustration of the system and method is shown. With this illustration that trim is processed in order to pump or push the product along conveyance channels that can then be scanned by the appropriate scanner as the product travels through a scan position. Once the product is scanned it can be separated and sorted to one of a plurality of product channels that are segregated by predefined percent ranges that are representative of the percent fat content desired for the given conveyance path.

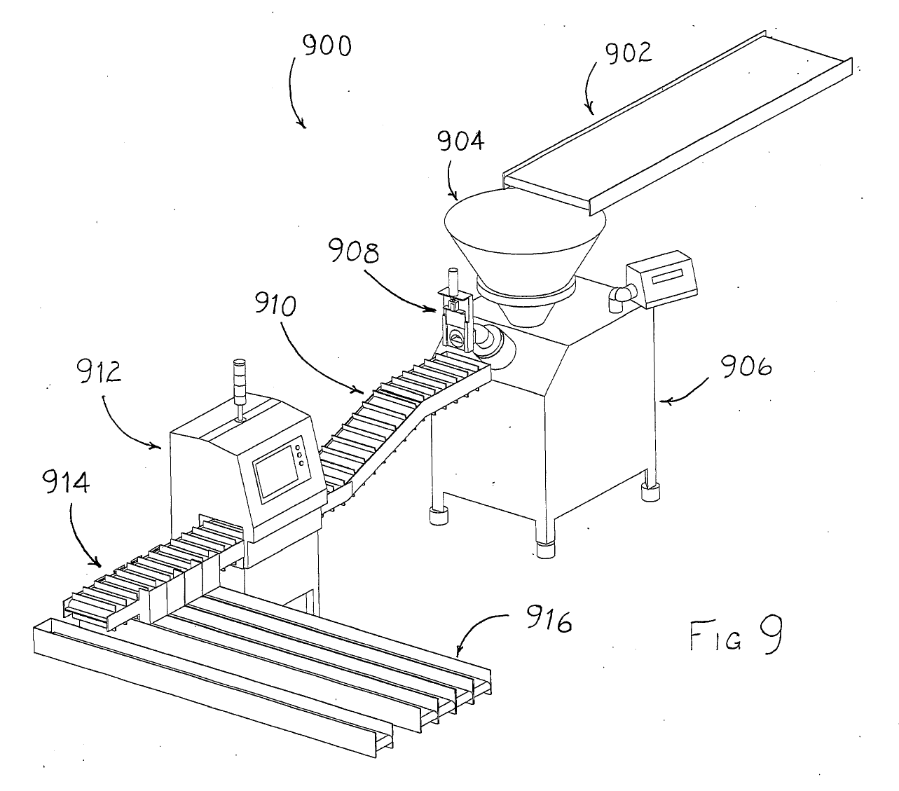

Referring to FIG. 9, an illustration of the system 900 having a flighted conveyor 910 is shown. An infeed conveyor 902 is illustrated, which can be utilized to convey the product toward and into the portioner hopper 904, which channel the product into the portioner 906, whereby the portioner can perform a pre-sizer/reducer function, which can reduce the product to presized chunks, which can be output to a flighted conveyor having flights timed to the output of the pre-sizer such that the presized chunks will fall between the flights. The portioner can have an output cutter 908, which is time to cut the product in sections and eject it to fall within one of the flights of the flighted conveyor 910. FIG. 9 provide an illustration of a single output cutter 908, however, multiple parallel outputs and cutters can be utilized within the scope of the invention. The scanner 912 can be positioned to scan the fat content of the chunks between the flights. The scan data can be captured by a controller and utilized to control a reject station or sorting device 914. The chunks are rejected at an appropriate rejection location based on percent fat content down a channeling chute, which channels the chunk onto a corresponding take-away conveyor 916.

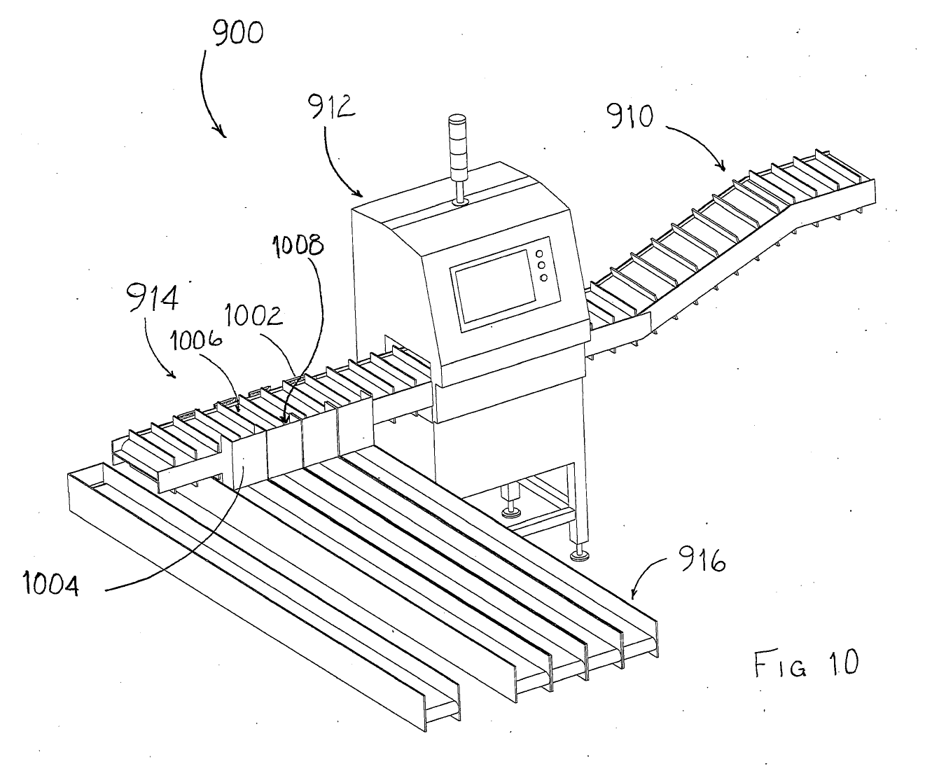

Referring to FIG. 10, an illustration of the system 900 having a flighted conveyor 910 and reject 914 is shown. The reject station can include spaced apart reject mechanisms 1002 positioned adjacent lanes of rejection 1006, which are inline with an opening 1008 to a channeling chute 1004, which is operable to channel the item onto a corresponding take-away conveyor. The spaced apart reject mechanisms, the corresponding lane of rejection, the corresponding channeling chute and the corresponding take-away conveyor can be segregated base on desired percent fat content. Therefore, when an item being conveyed and the flight in which it is contained has been conveyed to a position adjacent a reject mechanism and corresponding lane of rejection that has been assigned a correlating percent fat content, the item can be rejected laterally off the flighted conveyor by the rejection mechanism and down the corresponding channeling chute. A controller can be pre-programmed to assign control each rejection mechanism. The controller can also receive can also receive an input from the scanner for each item and can control the speed and timing of the flighted conveyor such that the controller knows the position of each item relative to all components including the cutter, scanner and reject mechanism. The item is channeled through the chute and onto a take away conveyor, which conveys the item away for further sorting and processing.

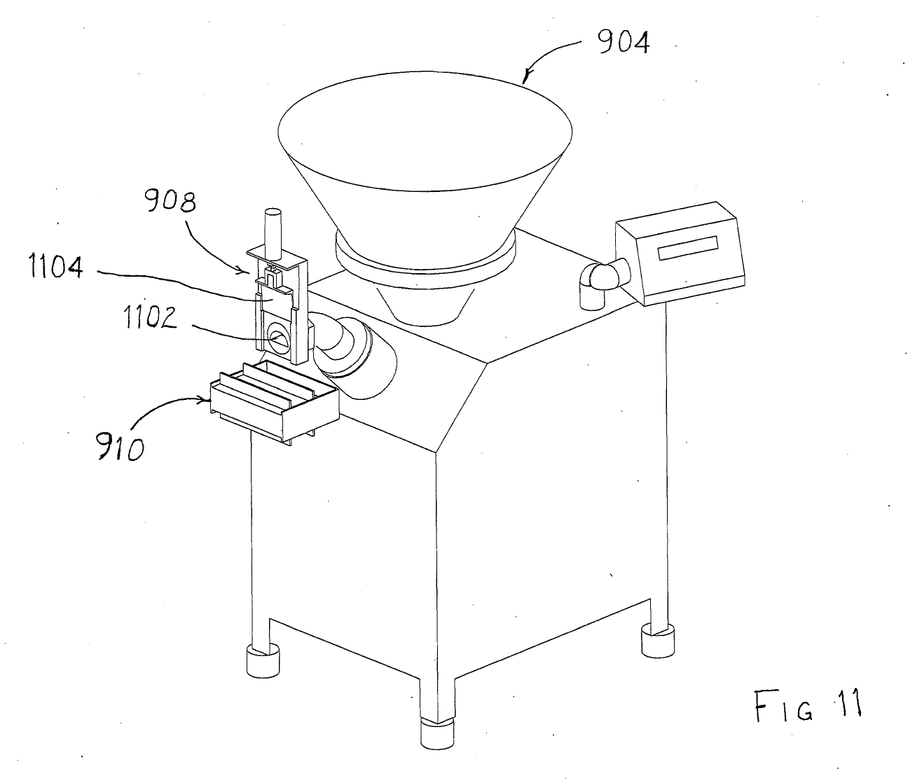

Referring to FIG. 11, an illustration of the system cutter and flighted conveyor is shown. The cutter 908 can be positioned at the exit end of the output port. The cutter 908 has a through channel 1102 through which the item is channeled for cutting. The cutter also has a cutting implement 1104, which can be controlled to cut at the appropriate time. The diameter size of the output port can be sized to control flow of items there through. When each item is separated by the cutter, the cutter can be positioned such that the item will fall to the flighted conveyor within on of the flights.

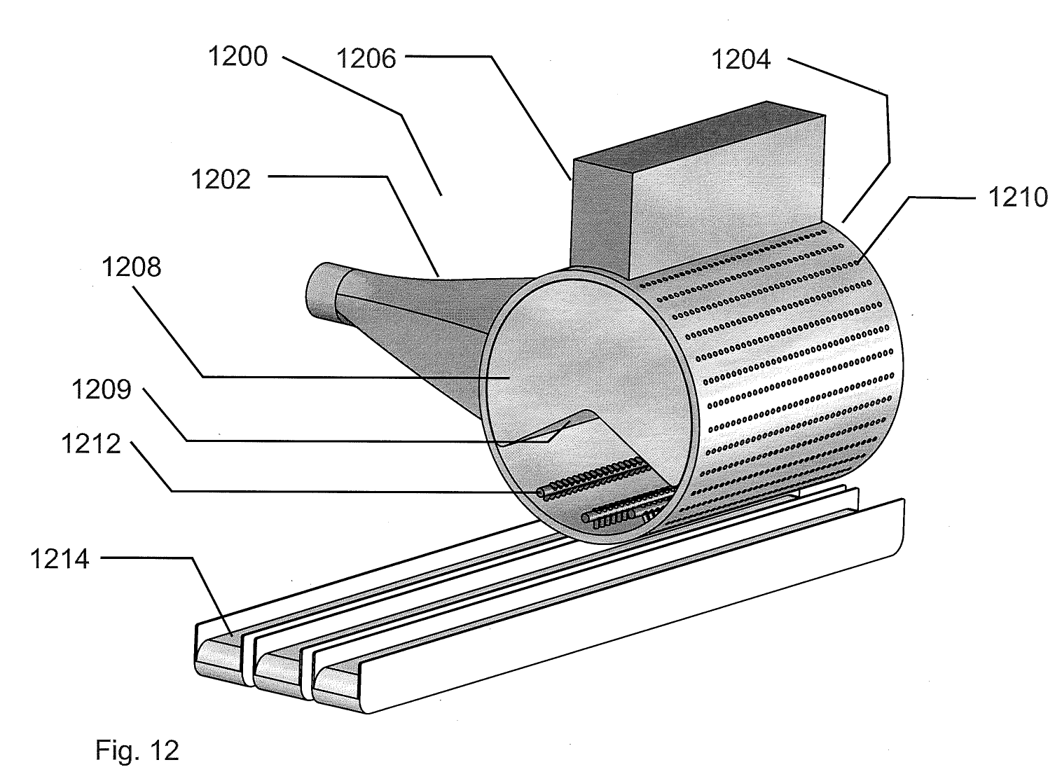

Referring to FIG. 12, an illustration of an embodiment utilizing a rotary portioning drum or drum cutter is shown. This embodiment of the system 1200 includes a distributor horn 1202 through which the product mass is pressed through or extruded under pressure. A pressurized side plate 1208 having a plate portion 1209 conforming to the interior of the portioning drum can keep the filled portioning form under pressure when it is being filled ensuring a consistent product shape and size that is being scanned. A product retention plate 1216 (not shown) can be utilized to hold the product in place as the rows of portioning forms travel along the lower rotation of the portioning drum. The distributor horn continuously distributes the product mass at a substantially constant rate and volume evenly over the portioning drum 1204 and thereby evenly filling the portioning forms 1210 as the drum continuously rotates pass the exit opening of the distributor horn. The portioning drum is shown having a plurality of rows of portioning forms 1210. The number of rows of portioning forms and the number of portioning forms per row may vary. The size and contour or shape of the portioning form may also vary. As the portioning drum rotates, the rows of portioning fauns can rotate pass a scanner 1206.

Again, as noted with the other embodiments disclosed herein, the scanner can be one of many technologies including NIR, X-ray, high-resolution digital photographic imaging, guided microwave spectroscopy system and various other digital and analog scanning systems. A scanning station 1206 can be disposed after the output of the distributor horn. The scanning system can be used to scan and detect fat content scan data in order to determine the combined fat analysis (FA) by every predefined scan length of the product within the portioning form. The scanner can provide an input to a controller system that can at the appropriate time initiate the scan, analyze the result, and selectively discharge the product within the portioning to the appropriate sorting conveyor 1214 based on the scan results. The discharged product will be dropped onto a conveyor 1214 that transports the product to a combining process or other process. The scanner software along with the controller can keep the aggregate FA of each of the products/conveyors (output streams). Each of the output streams can further be combined to get a desired out fat % combination.

The portioning drum, particularly the portioning forms of the portioning drum can be constructed of sintered stainless steel, through which air can permeate. The portioned product can be blown or ejected from the portioning form using air pressure. A plurality of air nozzles assemblies 1212 can be positioned along the lower rotation of the portioning drum. The air nozzles assemblies 1212 can be elongated bars having a plurality of individual ejection air nozzles and the air nozzle assembly 1212 can be fixedly mounted exterior of the drum and can extend into the interior of the portioning drum.

The air nozzle assemblies can be oriented to align above the rows of portioning forms as the rows travel along the lower rotation under the air nozzle assemblies. The air nozzle assemblies can be further positioned such that the individual ejection nozzles are positioned above a portion form within a row. Each individual ejection air nozzles can be individually and independently controlled. Each air nozzle assembly is position to eject formed product on to one of a plurality of sorting conveyors 1214. For a given row, a single air nozzle assembly will likely only eject a subset of formed product of the entire row onto a corresponding sorting conveyor based on the percent fat content of an individual formed product.

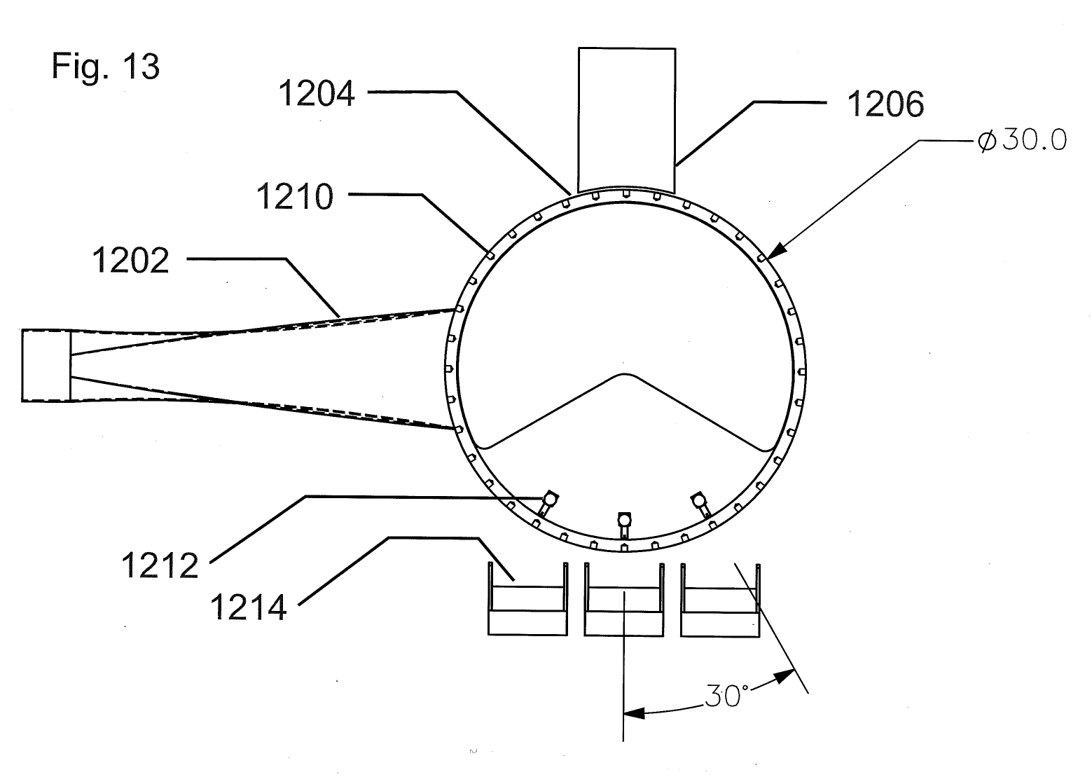

Referring to FIG. 13, an illustration of a side view of the rotary drum cutter is shown. The distributor horn 1202 is shown with its exit end immediately adjacent the portioning drum 1204. The portioning forms 1210 can rotate pass a scanner 1206. The side plate 1208 having a plate portion 1209 can conform to the interior of the portioning drum and can keep the filled portioning form under pressure. The portioning forms 1210 are filled as the drum continuously rotates pass the exit opening of the distributor horn 1202. The air nozzle assemblies 1212 are shown aligned with the rows as the rows travel along the lower end of the drum rotation. The formed product can be rejected and dropped onto a sorting conveyor 1214. A product retention plate 1216 (not shown) can be utilized to hold the product in place as the rows of portioning forms travel along the lower rotation of the portioning drum. However, the pressure of pumping the product into the cavity will trap the product until it gets blown out. There is a plate that is on the inside that the meat is pumped against and for ejections the air is blown through to kick it out of the cavity. Again, the number of portioning forms per row can vary and the number of rows can vary. Also, depending on the diameter of the portioning drum, the number of air nozzle assemblies and corresponding sorting conveyors may also vary.

The various lean recover examples shown above illustrate a novel method and apparatus for lean recovery from meat trimmings. A user of the present invention may choose any of the above lean recovery embodiments, or an equivalent thereof, depending upon the desired application. In this regard, it is recognized that various forms of the subject lean recovery method and apparatus could be utilized without departing from the spirit and scope of the present invention.

As is evident from the foregoing description, certain aspects of the present invention are not limited by the particular details of the examples illustrated herein, and it is therefore contemplated that other modifications and applications, or equivalents thereof, will occur to those skilled in the art. It is accordingly intended that the claims shall cover all such modifications and applications that do not depart from the sprit and scope of the present invention.

Other aspects, objects and advantages of the present invention can be obtained from a study of the drawings, the disclosure and the appended claims.

What is claimed is:

1. A method for segregating lean product based on percent fat content comprising the steps of;
pressing a lean product into and through a distributor horn and through an exit end of said distributor horn;
rotating a portioning drum having rows of exterior facing portioning forms adjacent said exit end;
distributing the lean product exiting the exit end over the portioning drum and filling the exterior facing portioning forms with the lean product;
scanning the lean product filled in the portioning forms;
analyzing the scan data and determining the percent fat content for each portioned lean product defined by the predetermined size of the lean product within a given portioning form;
and
sorting based on which of a plurality of defined fat content ranges the corresponding percent fat content is within and directing each portioned lean product segment down one of a plurality of processing paths corresponding to the one defined fat content range in which the corresponding percent fat content falls.

2. The method of segregating sparse lean product as recited in claim 1, where the scanner is a digital photographic imaging system.

3. The method of segregating lean product as recited in claim 1, where the scanner is a near infra-Red scanner.

4. The method of segregating lean product as recited in claim 1, where the predetermined size of the portioned product is approximately ½″ in diameter.

5. The method of segregating lean product as recited in claim 1, where the plurality of processing paths are a plurality of conveyor lanes.

6. The method of segregating lean product as recited in claim 5, where the portioned lean product is selectively ejected from the portioning form by an air nozzle to drop on one of a plurality of conveyor lanes based on percent fat content.

7. The method of segregating lean product as recited in claim 6, where each of the plurality of conveyor lanes has a corresponding air nozzle adapted to selectively emit air to selectively eject portioned lean product.

8. The method of segregating lean product as recited in claim 7, further comprising an encoder in electronic signal communication with the scanner and the scanner is adapted to provide an electronic control signal to control the air nozzle to selectively eject a portioned product.

9. The method of segregating lean product as recited in claim 8, further comprising the step of:
scanning each portioned product for segment fat content and segregating each portioned product further based on the portioned product's fat content.

10. The method of segregating lean product as recited in claim 8, further comprising the step of:
re-combining a combination of portioned product segments to achieve a desired recombined fat content.

11. A system for segregating lean product based on percent fact content comprising:
a portioning drum having exterior facing portioning forms and adapted to rotate such that the portioning forms pass adjacent an exit end of a distributor horn such that the portioning forms are finable with portioned lean product;
a scanner operable to scan the portioned lean product traveling pass the scanner and further operable to generate scan data representative of the percent fat content of the portioned lean product;
a processor electronically integrated with said scanner and operable to analyze the scan data and determine the percent fat content for each portioned lean product defined by the predetermined size of portioned lean product within the volume;
an encoder electronically integrated with the processor operable to control an air nozzle to selectively eject the portioned lean product; and
processing paths each corresponding to the one defined fat content range in which the corresponding percent fat content falls.

12. The system of segregating lean product as recited in claim 11, where the scanner is a photographic digital imaging system.

13. The system of segregating lean product as recited in claim 11, where the scanner is a near infra-Red scanner.

14. The system of segregating lean product as recited in claim 11, where the predetermined size is approximately ½″ in diameter.

15. The system of segregating lean product as recited in claim 11, where the plurality of processing paths are a plurality of conveyor lanes.

16. The system of segregating lean product as recited in claim 15, where each of the plurality of conveyor lanes has a corresponding air nozzle adapted to selectively emit air to selectively eject portioned lean product

17. The system of segregating lean product as recited in claim 16, where each of the air nozzles are adapted to selectively emit air jets to eject portion lean product onto a corresponding conveyor lane.

18. The system of segregating sparse lean product as recited in claim 17, where the encoder in electronic signal communication with the processor of the scanner and the scanner is adapted to provide an electronic control signal to control the air nozzle to selectively eject a portioned product.

19. The system of segregating sparse lean product as recited in claim 18, further comprising:
a second scanner operable to scan each sparse lean product segment for segment fat content; and
a segregator operable for segregating each sparse lean product segment further based on the segment fat content.

20. The system of segregating sparse lean product as recited in claim 19, further comprising the:
a combiner operable for recombining a combination of sparse lean product segments to achieve a recombined product to achieve a desired recombined fat content.

21. A system for segregating sparse lean product based on percent fact content comprising:
a pre-sizer having reduction end plate adapted to pre-size a sparse lean product;
an output port communicably attached to an output of the pre-sizer and said output port mounted as a conduit to receive the pre-sized sparse lean product and channel from an entry end to an exit end;
a cutter attached proximate the exit end of the conduit and adapted to cut away each segment;
a flighted conveyor disposed below the cutter having flight sections and having controlled timing to position the flight sections beneath the cutter to receive cut away segments in said flight sections and where said flighted conveyor is adapted to convey the received segments along a path of conveyance;
a scanner disposed along the path of conveyance and operable to scan cut away segments within the flights being conveyed along the path of conveyance and operable to generate scan data representative of the percent fat content of the ground sparse lean product segment;
a processor electronically integrated with said scanner and operable to analyze the scan data and determine the percent fat content for each product segment; and
an encoder electronically integrated with the processor and a reject mechanism operable control the reject mechanism to laterally eject the product segment out of the flight.

22. The system of segregating sparse lean product as recited in claim 21, where the reject mechanism corresponds to a predetermined percent fat content.

23. The system of segregating sparse lean product as recited in claim 22 further comprising:
a channeling chute positioned to receive the laterally rejected product segment, and which channels the segment onto a corresponding take-away conveyor.

24. The system of segregating sparse lean product as recited in claim 23, where said reject mechanism is an air jet nozzle and compressed air source operable to produce a jet of air sufficient to laterally eject the product segment.

25. The system of segregating sparse lean product as recited in claim 24, where the scanner is an X-Ray scanner.

26. The system of segregating sparse lean product as recited in claim 24, where the scanner is a near infra-Red scanner.

27. A method for segregating sparse lean product based on percent fact content comprising the steps of:
pre-sizing a sparse lean product with a pre-sizer having reduction end plate;
urging the pre-sized product out of an output port communicably attached to an output of the pre-sizer and said output port mounted as a conduit to receive the pre-sized sparse lean product and channeling the product from an entry end to an exit end of the conduit;
cutting the product into product segments with a cutter attached proximate the exit end of the conduit and adapted to cut away each segment;
capturing and conveying segments in flights a flighted conveyor disposed below the cutter having flight sections and controlling timing of the flighted conveyor to position the flight sections beneath the cutter to receive cut away segments in said flight sections and where said flighted conveyor is adapted for conveying the received segments along a path of conveyance;
scanning the segments with a scanner disposed along the path of conveyance and operable to scan cut away segments within the flights being conveyed along the path of conveyance and generating scan data with said scanner representative of the percent fat content of the ground sparse lean product segment;
analyzing the scan data with a processor electronically integrated with said scanner and operable to analyze the scan data and determining the percent fat content for each product segment; and
controlling a reject mechanism to laterally eject the product segment out of the flight with an encoder electronically integrated with the processor and the reject mechanism.

28. The method of segregating sparse lean product as recited in claim 27 further comprising the steps of:
channeling the laterally rejected product segment onto a corresponding take-away conveyor with a channeling chute positioned to receive the laterally rejected product segment and channel.

29. The method of segregating sparse lean product as recited in claim 28, where scanning is scanning with an X-Ray scanner.

30. The method of segregating sparse lean product as recited in claim 28, where scanning is scanning with a near infra-Red scanner.
